(19) United States
(12) Patent Application Publication
(10) Pub. No.: US 2004/0022791 A1
(43) Pub. Date: Feb. 5, 2004

(54) Recombined anti-gpiib/iiia antibodies used for inhibiting angiogenesis

(76) Inventors: Peter Berchtold, Hinterkappeln (CH); Robert Escher, Berne (CH)

Correspondence Address: ROTHWELL, FIGG, ERNST & MANBECK, P.C., 1425 K STREET, N.W., SUITE 800, WASHINGTON, DC, 20005, US

(21) Appl. No.: 10/399,701

(22) PCT Filed: Nov. 20, 2001
(86) PCT No.: PCT/EP01/13445

(30) Foreign Application Priority Data
Nov. 20, 2000 (DE) 100 57 443.2

(51) Int. Cl. A61K 39/395

(52) U.S. Cl. 424/155.1

(57) ABSTRACT

The present invention relates to the use of special phage-display-optimized antibodies directed against GPIIb/IIIa for jointly inhibiting fibrinogen binding to platelets and vitronectin binding to endothelial cells for the purpose of the therapy and/or prophylaxis of vascular occlusion. The present invention furthermore relates to the use of the antibodies for inhibiting angiogenesis and/or for inhibiting the metastasis of tumors and/or for inhibiting intima hyperplasia following vascular damage. bmmy 17.11.2000

### DESCRIPTION

The present invention relates to the use of special, phage-display-optimized antibodies directed against GPIIb/IIIa for jointly inhibiting fibrinogen binding to platelets and vitronectin binding to endothelial cells for the purpose of the therapy and/or prophylaxis of vascular occlusion. The present invention furthermore relates to the use of the antibodies for inhibiting angiogenesis and/or for inhibiting the metastasis of tumors and/or for inhibiting intima hyperplasia following vascular damage.

It has been known for a long time that glycoprotein IIb/IIIa (GPIIb/IIIa, also termed α_{IIb}β₃ or CD41/CD61) is expressed on the surface of thrombocytes. The receptor is also frequently termed the fibrinogen receptor since fibrinogen is the preferred ligand. However, in addition to this, the receptor also binds a large number of other ligands which contain the RGD sequence, such as fibronectin, vitronectin and von Willebrand factor.

It has also been known for a long time that GPIIb/IIIa plays an essential role in cellular hemostasis. Normally, thrombocytes do not remain attached to the vascular endothelium nor do they stick to each other. However, if the thrombocyte comes into contact with damaged blood vessels whose endothelium is torn, there then follows an interaction with the underlying matrix proteins, such as collagen, fibronectin or laminin, for which the thrombocyte possesses specific membrane receptors which are similar to the integrins. However, in blood vessels in which high shearing forces occur, these interactions are not sufficient for the platelets to adhere and for a clot to be formed. The latter becomes possible, inter alia, because the thrombocytes carry the glycoprotein IIb/IIIa (GPIIb/IIIa) on their surface, which protein mediates binding to fibrinogen and is therefore also termed the fibrinogen receptor. Following activation by intracellular messengers, the GPIIb/IIIa receptor recognizes fibrinogen molecules and in this way mediates crosslinking of the thrombocytes (Löffler & Petrides, Biochemie und Pathobiochemie [Biochemistry and Pathobiochemistry], Springer, Berlin).

Germ line mutations in the genes for the GPIIb/IIIa receptor lead to a rare, autosomally recessive bleeding disease which is characterized by a prolonged bleeding time, normal thrombocyte values and the complete absence of platelet aggregation and is termed Glanzmann's thrombasthenia (Löffler & Petrides, Biochemie und Pathobiochemie, Springer, Berlin).

Conversely, it is possible to block the GPIIb/IIIa receptor for the purpose of preventing or treating undesirable vascular occlusions. Small molecules or else antibodies are suitable for this purpose. Abciximab (ReoPro) is a human/mouse chimeric monoclonal antibody Fab fragment which is derived from the murine monoclonal antibody 7E3 and which binds with great avidity both to the activated and the non-activated form of GPIIb/IIIa. This antibody has been licensed as supplementary therapy for preventing ischemic complications in the heart in patients who undergo a percutaneous intervention in the coronary blood vessels. Other indications for abciximab (ReoPro) are unstable angina, stenting in the carotid, ischemic stroke and peripheral vascular diseases (Cohen et al., Pathol. Oncol. Res. 6: 163-174 (2000)).

Cell types which [lacuna] GPIIb/IIIa and α₅β₃ are also thought to be connected with events in angiogenesis, vascularization and neovascularization. Hypoxia, as occurs, for example, in diabetes, asthma and Alzheimer's disease, and inflammatory processes are considered to be inducers of the angiogenesis. Thereby in many cases, this angiogenesis is desirable, for example because it counteracts ischemia following myocardial infarctions and ensures that the heart tissue is supplied with oxygen and other essential factors. However, since the blood vessels which are subsequently formed are frequently not fully differentiated, the subsequent formation of the blood vessels can also be undesirable. A list of diseases which are associated with increased vascularization has recently been published (Carmeliet & Jain, Nature 407 (2000), 249-257).

In any case, tumor angiogenesis is undesirable. As early as 1971, it was postulated that tumor growth and metastasis proceed in an angiogenesis-dependent manner (Folkman, J. Cancer Medicine (eds. Holland, J. F. et al.), 132-152). This made it clear that inhibiting angiogenesis was a possible strategy for inhibiting tumor growth and metastasis. Tumor blood vessels differ from normal blood vessels as a result of their irregular construction, variable diameter and excessive ramifications and openings between the endothelial cells delimiting the blood vessel and a discontinuous or absent basal membrane (Carmeliet & Jain, Nature 407 (2000), 249-257). Although at least some tumor blood vessels possess a mosaic-like construction composed of endothelial cells and cancer cells, it remains difficult to specifically recognize tumor-specific blood vessels. It is true that it has been possible, by selecting phage-display libraries in vivo, to isolate peptides which preferentially recognize the blood vessels of subcutaneous tumors in mice (Arap et al., Science 279 (1998), 377-380). However, it has still not been demonstrated that these peptides can be used in vivo to effectively concentrate antineoplastic agents in the region of the tumor. For this reason, most efforts are still being directed toward developing angiogenesis inhibitors for the purpose of treating tumors. A current list of the substances tested in clinical experiments can be found in Carmeliet (Carmeliet & Jain, Nature 407 (2000), 249-257).

However, molecules directed against GPIIb/IIIa are not only of interest for controlling the growth and metastasis of tumors, on account of their angiogenesis-inhibiting properties. Thus, it has become clear, as a result of more recent studies, that expression of the GPIIb/IIIa receptor is not restricted to thrombocytes. Thus, Trikha et al. showed, both at the RNA level and the protein level, that the human. melanoma cell lines WM983B, WM983A and WM35 express GPIIb/IIIa (Trikha et al., Cancer Res. 57: 2522-2528 (1997)). Timar et al. have also obtained similar results in the case of B16a metastatic melanoma cells. These authors also showed that stimulating cells with a protein kinase C activator stimulates the translocation of GPIIb/IIIa from an intracellular pool to the cell surface. The expression of GPIIb/IIIa plays a role in cell adhesion and, in particular, in tumor cell invasion through the basal membrane.

In WO 98/55619, the applicant recently described improved antibodies directed against GPIIb/IIIa, which antibodies were optimized by means of phase display and panning selection. The disclosure in this patent specification contains detailed information with regard to the hypervariable, complementarity-determining regions (CDRs) and the framework regions (FRs).

The object of the present invention was to search for further indications for the antibodies described in WO 98/55619.

This object was achieved by the surprising observation that the antibodies can be used to jointly inhibit fibrinogen binding to platelets and vibronectin binding to endothelial cells, which means that these antibodies are particularly well suited for treating and/or preventing vascular occlusion. The inhibition of the binding of vitronectin to endothelial cells may possibly be connected to the expression of the vitronectin receptor α₅β₃ on endothelial cells. It is assumed that the antibodies from WO 98/55619 crossreact with the vitronectin receptor on endothelial cells, and in this way inhibit the binding of vitronectin, on account of the subunit β₃ being possessed in common. However, it is also possible that the effect is mediated by other integrin receptors which recognize the RGD sequence or whose ligand binding can be inhibited by RGD. Because of the fact that the antibodies crossreact with the vitronectin receptor α₅β₃ on endothelial cells, the antibodies can also be used for treating intimahyperplasia following vascular damage. This indication for the WO 98/55619 antibodies follows from the observation that α₅β₃ (=CD51/CD61) is connected with intimahyperplasia following vascular damage.

It has furthermore been found, surprisingly, that the antibodies described in WO 98/55619 possess very promising properties which make it appear appropriate to use these antibodies for inhibiting angiogenesis. The antibodies are consequently suitable for treating tumors and, in particular, for preventing the metastasis of tumors since they impede the vascularization of the primary tumor and consequently impede its ability to colonize surrounding tissue and release proliferating cells into the blood circulation. In addition, it must be assumed that blocking the GPIIb/IIIa receptors with the antibodies described in WO 98/55619 impairs the ability of migrated tumor cells to adhere at the target site and penetrate into the tissue.

The invention accordingly relates to the use of the heavy chain of an antibody, of a functional derivative, or of a fragment thereof, comprising a CDR3 region selected from:

(a) an amino acid sequence:

V L P F D P I S M D V (I)

(b) an amino acid sequence:

A L G S W G G W D H Y M D V (II)

(c) an amino acid sequence having a homology of at least 80% with an amino acid sequence from (a) or (b)

(d) an amino acid sequence having an equivalent ability to bind to GPIIb/IIIa

for inhibiting angiogenesis and/or for inhibiting the metastasis of tumors and/or for inhibiting intimahyperplasia following vascular damage.

The heavy chain according to the invention, the functional derivative or the fragment thereof preferably furthermore comprises a CDR1 region selected from:

(a) an amino acid sequence:

G Y S W R (III)

(b) an amino acid sequence:

S Y A M H (IV)

(c) an amino acid sequence which exhibits a homology of at least 80% with an amino acid sequence from (a) or (b).

The heavy chain according to the invention, the functional derivative or the fragment thereof furthermore preferably comprises a CDR2 region selected from:

(a) an amino acid sequence:

D I S Y S G S T K Y K P S L R S (V)

(b) an amino acid sequence:

V I S Y D G S N K Y Y A D S V K G (VI)

(c) an amino acid sequence which exhibits a homology of at least 80% with an amino acid sequence from (a) or (b).

A further aspect of the present invention is accordingly the use of the light chain of an antibody, of a functional derivative or of a fragment, selected from:

(a) an amino acid sequence:

A T W D D G L N G P V (VII)

(b) an amino acid sequence:

A A W D D S L N G W V (VIII)

(c) an amino acid sequence which exhibits a homology of at least 80% with an amino acid sequence from (a) or (b), and

(d) an amino acid sequence having an equivalent ability to bind to GPIIb/IIIa

for inhibiting angiogenesis and/or for inhibiting the metastasis of tumors and/or for inhibiting intimahyperplasia following vascular damage.

The light chain according to the invention, the functional derivative or the fragment thereof furthermore preferably comprises a CDR1 region selected from:

(a) an amino acid sequence:

S G S S S N I R S N P V S (IX)

(b) an amino acid sequence:

S G S S S N I G S N T V N (X)

(c) an amino acid sequence having a homology of at least 80% with an amino acid sequence from (a) or (b).

In addition, the light chain according to the invention, the functional derivative or the fragment thereof preferably furthermore comprises a CDR2 region selected from:

(a) an amino acid sequence:

G S H Q R P S (XI)

(b) an amino acid sequence:

S N N Q R P S (XIII)

(c) an amino acid sequence having a homology of at least 80% with an amino acid sequence from (a) or (b)

Within the meaning of the present invention, the expression “functional derivative of a chain of a human antibody” is to be understood as meaning a polypeptide which comprises at least a CDR3 region of the heavy and/or light chain as defined above and, together with the respective complementary chain of the human antibody (or a derivative of such a chain), can form an antibody derivative which possesses a recognition specificity for an antigen which is equivalent to that of the underivatized antibody. Preferably, such an antibody derivative has a binding constant of at least 10⁶ l/mol, preferably of at least 10⁸ l/mol, for the given antigen.

Functional derivatives of chains of a human antibody can be prepared, for example, by using recombinant DNA techniques to delete, substitute and/or insert segments of the gene encoding the given polypeptide.

Particularly preferred functional derivatives of antibody chains or antibodies are single-chain antibodies which can be assembled, for example, from the variable domains of the H chain and the L chain and, where appropriate, a constant domain. The preparation of such constructs is described in Hoogenboom et al., Immunol. Rev. 130 (1992), 41-68; Barbas III, Methods: Companion Methods Enzymol. 2 (1991), 119 and Plückthun, Immunochemistry (1994), Marcel Dekker Inc., Chapter 9, 210-235.

Within the meaning of the invention, the expression “equivalent binding ability” is to be understood as meaning an identical binding affinity and/or specificity, i.e. epitope recognition as in the specifically disclosed sequences.

The present invention also relates to the use of a vector, which contains at least one copy of a nucleic acid which encodes one of the above-described antibodies, for inhibiting angiogenesis and/or for inhibiting the metastasis of tumors and/or for inhibiting intimahyperplasia following vascular damage. Treating the patient with nucleic acid instead of protein has a number of advantages. Whereas storing protein is a relatively elaborate matter, DNA can be stored without difficulty even over long periods of time. Another advantage of using DNA is that the antibodies are correctly processed posttranslationally, for example with regard to glycosylation.

Independently of the administration form, use, according to the invention, of the abovementioned antibodies possesses substantial advantages as compared with the previously known use of the ReoPro Fab fragment. These advantages include the fact that the abovementioned antibodies comprise amino acid sequences which are entirely of human origin and the danger of an undesirable immune reaction against the antibodies which are employed is consequently kept as low as possible.

1. The use of the heavy chain of an antibody, of a functional derivative, or of a fragment thereof, comprising a CDR3 region selected from:
(a) an amino acid sequence:
V L P F D P I S M D V (I)
(b) an amino acid sequence:
A L G S W G G W D H Y M D V (II)
(c) an amino acid sequence having a homology of at least 80% with an amino acid sequence from (a) or (b)
(d) an amino acid sequence having an equivalent ability to bind to GPIIb/IIIa
(1) for jointly inhibiting fibrinogen binding to platelets and vitronectin binding to endothelial cells
(2) for inhibiting angiogenesis and/or
(3) for inhibiting the metastasis of tumors and/or
(4) for inhibiting intimahyperplasia following vascular damage.

2. The use as claimed in claim 1, wherein the heavy chain, the functional derivative or the fragment thereof furthermore comprises a CDR1 region selected from:
(a) an amino acid sequence:
G Y S W R (III)
(b) an amino acid sequence:
S Y A M H (IV)
(c) an amino acid sequence which exhibits a homology of at least 80% with an amino acid sequence from (a) or (b).

3. The use as claimed in claim 1 or 2, wherein the heavy chain, the functional derivative or the fragment thereof furthermore comprises a CDR2 region selected from:
(a) an amino acid sequence:
D I S Y S G S T K Y K P S L R S (V)
(b) an amino acid sequence:
V I S Y D G S N K Y Y A D S V K G tm (VI)
(c) an amino acid sequence which exhibits a homology of at least 80% with an amino acid sequence from (a) or (b).

4. The use of the light chain of an antibody, of a functional derivative, or of a fragment thereof, comprising a CDR3 region selected from:
(a) an amino acid sequence:
A T W D D G L N G P V (VII)
(b) an amino acid sequence:
A A W D D S L N G W V (VIII)
(c) an amino acid sequence which exhibits a homology of at least 80% with an amino acid sequence from (a) or (b), and
(d) an amino acid sequence having an equivalent ability to bind to GPIIb/IIIa
(1) for jointly inhibiting fibrinogen binding to platelets and vitronectin binding to endothelial cells
(2) for inhibiting angiogenesis and/or
(3) for inhibiting the metastasis of tumors and/or
(4) for inhibiting intimahyperplasia following vascular damage.

5. The use as claimed in claim 4, wherein the light chain, the functional derivative or the fragment thereof furthermore comprises a CDR1 region selected from:
(a) an amino acid sequence:
S G S S S N I R S N P V S (IX)
(b) an amino acid sequence:
S G S S S N I G S N T V N (X)
(c) an amino acid sequence having a homology of at least 80% with an amino acid sequence from (a) or (b).

6. The use as claimed in claim 4 or 5, wherein the light chain, the functional derivative or the fragment thereof furthermore comprises a CDR2 region selected from:
(a) an amino acid sequence:
G S H Q R P S (XI)
(b) an amino acid sequence:
S N N Q R P S (XIII)
(c) an amino acid sequence having a homology of at least 80% with an amino acid sequence from (a) or (b).

7. The use of an antibody, of a functional derivative or of a fragment thereof, comprising
(a) a heavy chain, a functional derivative or a fragment thereof as in one of claims 1 to 3
(b) a light chain, a functional derivative or a fragment thereof as in one of claims 4 to 6
(1) for jointly inhibiting fibrinogen binding to platelets and vitronectin binding to endothelial cells
(2) for inhibiting angiogenesis and/or
(3) for inhibiting the metastasis of tumors and/or
(4) for inhibiting intimahyperplasia following vascular damage.

8. The use as claimed in one of claims 1 to 7 for the therapy and/or prophylaxis of vascular occlusion.

9. The use as claimed in one of claims 1 to 7 for tumor therapy.

10. The use of nucleic acid which encodes a protein as in one of claims 1 to 9
(1) for jointly inhibiting fibrinogen binding to platelets and vitronectin binding to endothelial cells
(2) for inhibiting angiogenesis and/or
(3) for inhibiting the metastasis of tumors and/or
(4) for inhibiting intimahyperplasia following vascular damage.

11. The use as claimed in claim 10 for the therapy and/or prophylaxis of vascular occlusion.

12. The use as claimed in claim 10 for tumor therapy.


SEQUENCE LISTING (12 sequences)

SEQ ID NO: 1 - protein, 11 aa, Artificial Sequence
  Description of Artificial SequenceCDR3-region of an optimized antibody against GPIIB/IIIA
VLPFDPISMDV

SEQ ID NO: 2 - protein, 14 aa, Artificial Sequence
  Description of Artificial Sequence CDR3-region of an optimized antibody against GPIIB/IIIA
ALGSWGGWDHYMDV

SEQ ID NO: 3 - protein, 5 aa, Artificial Sequence
  Description of Artificial Sequence CDR1-region of an optimized antibody against GPIIB/IIIA
GYSWR

SEQ ID NO: 4 - protein, 5 aa, Artificial Sequence
  Description of Artificial Sequence CDR1-region of an optimized antibody against GPIIB/IIIA
SYAMH

SEQ ID NO: 5 - protein, 16 aa, Artificial Sequence
  Description of Artificial Sequence CDR2-region of an optimized antibody against GPIIB/IIIA
DISYSGSTKYKPSLRS

SEQ ID NO: 6 - protein, 17 aa, Artificial Sequence
  Description of Artificial Sequence CDR2-region of an optimized antibody against GPIIB/IIIA
VISYDGSNKYYADSVKG

SEQ ID NO: 7 - protein, 11 aa, Artificial Sequence
  Description of Artificial Sequence light chain of an optimized antibody against GPIIB/IIIA
ATWDDGLNGPV

SEQ ID NO: 8 - protein, 11 aa, Artificial Sequence
  Description of Artificial Sequence light chain of an optimized antibody against GPIIB/IIIA
AAWDDSLNGWV

SEQ ID NO: 9 - protein, 13 aa, Artificial Sequence
  Description of Artificial Sequence CDR1-region of an optimized antibody against GPIIB/IIIA
SGSSSNIRSNPVS

SEQ ID NO: 10 - protein, 13 aa, Artificial Sequence
  Description of Artificial Sequence CDR1-region of an optimized antibody against GPIIB/IIIA
SGSSSNIGSNTVN

SEQ ID NO: 11 - protein, 7 aa, Artificial Sequence
  Description of Artificial Sequence CDR2-region of an optimized antibody against GPIIB/IIIA
GSHQRPS

SEQ ID NO: 12 - protein, 7 aa, Artificial Sequence
  Description of Artificial Sequence CDR2-region of an optimized antibody against GPIIB/IIIA
SNNQRPS